(19) United States
(12) Patent Application Publication
(10) Pub. No.: US 2018/0240547 A1
(43) Pub. Date: Aug. 23, 2018

(54) Healthcare Visit Value Calculator

(71) Applicant: Upfront Healthcare Services, Inc., Evanston, IL (US)

(72) Inventor: Benjamin Albert, Evanston, IL (US)

(73) Assignee: Upfront Healthcare Services, Inc.

(21) Appl. No.: 15/903,329

(22) Filed: Feb. 23, 2018

Related U.S. Application Data
(60) Provisional application No. 62/462,525, filed on Feb. 23, 2017.

(51) Int. Cl. G16H 40/20; G06Q 10/06; G16H 80/00

(52) U.S. Cl. CPC G16H 40/20; G16H 10/60; G16H 80/00; G06Q 10/0631

(57) ABSTRACT

A healthcare visit value calculation system wherein a processor coupled with a memory executes computerized code which analyzes data regarding patients, doctors, and financial information to generate a visit value score. The system will then notify end users of this visit value score, monitor the score, and attempt to improve the score via automated and manual intervention.

### BACKGROUND

The present subject matter relates generally to a system, method, and apparatus for obtaining healthcare data across diverse data sets, analyzing and processing the data, and utilizing the data to optimize healthcare services and outcomes. More specifically, the present invention relates to a computerized system for monitoring and analyzing healthcare patient, provider, and institutional data and a set of rules to be used therein.

A significant problem encountered by healthcare providers relates to determining how to maximize the value of a visit (to them or the patient) given numerous potential factors across diverse and varying datasets, ranging from financial factors to patient-outcome factors. These complex patient, provider, and institutional variables can fluctuate from visit to visit and program to program. As set forth in more detail below, existing technological solutions to this problem rely on system architectures that do not allow access to and consideration of the full breadth of relevant datasets, and for instance do not allow application of rules derived from broad data sources and contracts-related information to individual healthcare practices.

In recent years, healthcare reimbursement for ambulatory (outpatient) care has begun to move from “fee for service” model to a “value-based” model. Value-based models of reimbursement tie revenue to more than just clinical services rendered. In value-based care, it is common for revenue to also be tied to, for example, quality of care metrics or patient satisfaction survey scores. In addition to value-based reimbursement, there are additional healthcare trends such as high deductible health insurance plans, a scarcity of primary care physicians, movement to providing care through computerized telehealth applications, and utilizing clinicians other than physicians (e.g., Nurse Practitioners). All of these healthcare trends create a new, more complex technical environment for determining the financial and clinical value of a visit to the patient, the providing clinician, and the providing institution. Computerized systems are uniquely useful in analyzing the wealth of data associated with all factors impacting value of a visit.

Computerized systems, through deterministic algorithms and machine learning, are also a powerful way to identify the key factors that contribute to visit value of each individual visit or aggregated visit by clinician, location, or institution. Using predictive analytics approaches, computerized systems can detect opportunities to improve the visit before it occurs and rank most impactful interventions to enhance visit value. Those impactful interventions can then be suggested to patients, clinicians, or other appropriate staff to maximize the visit value in advance of the clinical encounter.

Prior art approaches, however, have either not recognized, or not realized, these potential benefits from the use of computerized systems in the evaluation and utilization of visit value information for ambulatory healthcare visits. One significant challenge encountered in the computerized scheduling and valuation systems, is the need to gather data from multiple diverse data sets and to process that data in a manner that is usable for optimizing the clinical encounter. Prior art systems available in healthcare environments typically lacked connections to all of the sources of data—including servers, databases, and other local or remote repositories—pertaining to each of the relevant factors. For example, a prior art system may have had access to certain local patient records stored at a doctor's office, but lacked access or connections to servers or databases containing information relating to national surveys of healthcare outcomes, information about insurance contracts, or patient demographic information. Relatedly, prior art approaches typically processed, at most, a single data source in isolation, using a processing engine that was situated locally to—or in communication with—only a single data source. Thus, prior art approaches were unable to identify and resolve tensions between competing goals represented by competing metrics, such as physician utilization, physician and patient financial considerations, and patient scheduling. Further, the patient, the individual physician, the office clinical staff, the clinic, clinical department, location, and overall enterprise are all potential beneficiaries of improved healthcare-visit value, but each of these entities has different perspectives and priorities. The present invention allows all of these actors to be aligned and coordinated with the patient to ensure the visit is valuable for both parties, and time is not wasted by the patient or clinical provider.

Yet another issue with existing technologies is that these systems rarely account for existing contracts with insurers that set reimbursement rates for services, goods, and incentive payment goals for quality of care. These are all specific to each provider and can vary substantially in levels of payment and incentive structures. No current system incorporates or synthesizes such data in tandem with information regarding various complex data points which impact the value of a given visit. Furthermore, existing systems do not allow users to select areas of focus—for example, one provider may be more interested in more effective use of non-physician providers (e.g., nurse practitioners), while another is more interested in reducing patient no-show rate. Such options provide a highly customizable output for optimizing a visit to each healthcare provider and the ability to analyze data so that each provider may learn from broader practice.

Accordingly, there is a need for a system, method, and apparatus for monitoring, analyzing, and optimizing healthcare services.

### BRIEF SUMMARY OF THE INVENTION

To meet the needs described above and others, the present disclosure provides a system, method, and apparatus for monitoring, analyzing, and optimizing healthcare services.

The present solution provides an analytics engine that is connected to provider-specific data, nationwide healthcare outcome data, national census data, insurance, contracts, and financial datasets, that overcomes these problems and allows a practitioner to identify key areas where intervention will maximize patient, clinical, operational and financial value for each ambulatory patient visit. The system of the present invention may apply specific types of deterministic and probabilistic rules to provide actionable information about the likely value of a healthcare visit—information that was previously unavailable or, at best, available only in a limited form that often required human guesswork. The rules are generated from a variety of statistical techniques including machine learning approaches. The inventive system can, for example, create an optimized list of intervention opportunities and present the information to appropriate personnel. This approach enables proactive action to optimize the patient's healthcare visit in advance of the appointment occurring.

The invention may comprise a unique architecture of hardware and software (including, without limitation servers, databases, and analytic engines), and may further comprise a set of rules or processes designed to the purposes of the invention.

Our invention is designed to account for these challenges and solve them in a manner that provides significant value to physicians, patients, healthcare organizations, and third-party payors. In addition, our computational approach predicts, and assists in optimizing, the clinical encounter while there is time to intervene to improve the value. Our invention may comprise a rules engine which, upon receiving, analyzing, and processing data, identifies and ranks specific opportunities to improve the clinical encounter. In certain embodiments, the rules engine is able to dynamically adjust its outputs based on the analysis of data gathered from patient, physician, or third-party interactions. While there may be some computerized systems that currently assign a value to a given healthcare visit, these existing technologies focus on a single or limited set of variables in establishing such a value (e.g., cost of providing visit or revenue generated). This limited scope of existing systems does not utilize or synthesize all relevant and important data sources (some of which are not typically available to the existing systems) to both analyze and then optimize the visit. Additionally, existing technologies do not predict the impact on overall visit value by specific intervention. For example, moving a patient to see a nurse practitioner in 3 days rather than a physician in 3 weeks for addressing a specific clinical issue would be expected to yield significant clinical benefits to the patient and operational benefits the healthcare provider. Clinical and operational benefit would be expected because moving the appointment up would increase patient satisfaction at being seen in a timely fashion, avoid lost staff/facility costs by increasing the likelihood of the patient attending the appointment, and free up a physician to see a new patient or more complex patient (thus generating additional revenue and making more efficient use of the physician's time). The magnitude of the anticipated benefits is ranked for each upcoming appointment, allowing healthcare providers to select specific interventions they wish to implement to maximize expected financial and clinical outcomes.

In one embodiment, the system may be a computing system comprising a network of interconnected servers consisting of, for example, one or more of a data collection engine, integration engine, processing engine, analytics engine, application programming interface (API) engine, and a User Interface (UI) engine. As would be understood by a person of skill in the art, a “server” as used herein may refer to a physical server or to a virtual server, which may comprise a virtual machine or other appropriate virtualization software operating on one or more computer processors. As a non-limiting example, a virtual server may comprise virtualization software running on a web server provided as part of the “cloud” by Microsoft Azure, Amazon Web Services, or a similar provider. More than one virtual server may run on the same processor or set of processors, and a single virtual server may be distributed among more than one processor. Further, as used herein, an “engine” typically refers to a server or servers executing software stored in a nontransitory computer-readable medium.

The invention as disclosed herein may consist of one or more data collection engines. The data collection engines may be operatively connected to data sources in order to identify and gather data that can be used for patient care analysis and/or optimization. These data sources may comprise servers or other computer hardware comprising relational databases or other logical or physical structures in which data is stored in, and may be accessed from, a non-transitory computer-readable storage medium. Data sources may also comprise, in some embodiments, a user interface that is adapted to provide the system with information communicated to it by an individual physician, other healthcare professional, payer, or patient.

For example, data collection engines as contemplated by the invention may obtain data transmitted by health care providers related to healthcare contract terms, insurance coverage, patient treatment histories, patient payment needs, fee for value programs, and healthcare provider outcomes. Such data may be useful to generate advanced predictive analytics which help practices not only predict issues, but analyze and resolve them in the most efficient manner possible. The data collection engine may also gather data from sources that are not traditionally used for scheduling but could be considered for purposes of analyzing and/or optimizing a healthcare visit. For example, the data collection engine may gather income or other data that demonstrates a patient's ability to pay for services.

The invention as disclosed herein may further comprise an integration engine. The integration engine is communicatively connected to one or more data collection engines, and may also (or alternatively) be communicatively connected to one or more data sources. The integration engine may be connected to any data sources that a data collection engine may be collected to, as described above. Where the integration engine is communicatively coupled to a data source without the presence of an intervening data engine, the integration engine provides the functionality of the data collection engine with respect to that data source.

The integration engine may be communicatively coupled to data collection engines or data sources over a local or wide-area network, including the Internet, or through a proprietary or dedicated point-to-point data connection. The integration engine may also be coupled to one or more local data sources or local data collection engines, which may be accessed without communication over a network. The protocols used to accomplish such connections are well known in the art. The data sources and data collection engines to which the integration engine is coupled may include generally-applicable sources, containing data of interest to multiple healthcare practices, and may include sources specific to an individual healthcare practice (such as information relating to transportation options to that practice, or to an individual patient of that practice). The data sources or data collection engines communicatively connected to the integration engine may, in various embodiments of the invention, include sources providing one or more of the following: revenue-cycle data, billing data, payer data, quality-program data, patient demographic data, healthcare staff data, national healthcare datasets, contract- or insurance-related data, and patient history or personal data.

The system distributes computational tasks to optimize performance for required processes to optimize patient visits and calculate overlying analytics. This centralized server network may communicate with various end users and external databases to collect and analyze healthcare data related to patients, payers, quality programs, and doctors. From this analysis of data, the system may calculate and optimize the value of various visits scheduled between a provider and their patients and help the clinician optimize each visit.

The system may also consist of one or more processing engines, which may be communicatively coupled to the integration engine. FIG. 13 illustrates the processing engine in a non-exhaustive fashion. Other components and tasks may be a part of the processing engine. The processing engine may be communicatively connected to the integration engine and can access the data that is integrated by the integration engine. The processing engine may then transform the data received from the integration engine into usable formats by transforming, interpreting, converting, and aggregating the data. The processing engine may also use or create “metadata” from the raw data received from the integration engine. For instance, if raw patient data indicates that a patient has had ten healthcare visits over the past year and presented with different symptoms in each, metadata may be created to indicate that this may be a “complex” patient requiring a longer appointment or attention from a specialized physician.

The system may also consist of a statistical or analytics engine communicatively connected to the processing engine. The analytics engine accesses data provided by the processing engine, and uses that data to generate and apply analytical rules to be used in calculating a visit value score for individual patient visits. A visit value score is intended to reflect the value (either in monetary terms, clinical outcomes, patient satisfaction, completion of the appointment, time/day of the appointment, or in a different metric supplied by the provider) of a specific scheduled patient visit. In other words, the visit value score is calculated to reflect the “revenue” (or other financial value) of the visit, less costs, with additional value calculations dependent on clinical outcomes, operational factors including time/day lot utilized, strategic value (e.g., high priority strategic partner, developing clinical department), and the probability of the patient showing up for the appointment. This score is calculated retrospectively. In another embodiment, the visit value score can be predicted in advance of the visit, using predictive statistical techniques, including machine learning approaches or human-generated heuristics. To the extent that the prior art attempted to calculate visit values at all, all or some of the steps required manual intervention and/or were done without access to all of the sources of data described in this invention. Further, in the limited efforts in the prior art to calculate metrics of visit value, the software engines responsible for calculating such metrics were located local to healthcare providers and generally lacked connections to the data sources and data processing engines described herein. By locating the analytics engine as described in the present invention, the invention allows for the analytics engine to integrate a wider and more diverse set of information to calculate visit values for an individual healthcare provider. As one example, the present invention allows the analytics engine to utilize information that extends beyond the patient population seen by an individual healthcare provider, and rules derived from such information, to calculate visit value scores for that provider. The system architecture of the present invention also allows the analytics engine to utilize contract-related information or other information relating to patients' insurance information, which was not typically available in prior art systems. Finally, the architecture of the present invention allows the analytics engine to balance and evaluate competing value considerations for a given appointment, such as balancing payment-related considerations with considerations relating to the likelihood of a patient's appearing for his appointment. Prior-art systems, to the extent they evaluated visit value at all, lacked the connected architecture of the present invention and therefore considered these competing concerns in isolation (or not at all).

Returning to the visit value calculation of the analytics engine of the present invention, the analytics engine calculates visit value scores using both deterministic and probabilistic rules. Deterministic rules are ones where a given outcome is essentially known from a given set of inputs. For instance, a deterministic rule may indicate that, if a patient needs to have procedure X performed, the cost to the provider of materials and healthcare provider time needed for that procedure will be $500. Another deterministic rule may indicate that, if a patient is appearing for treatment Y, but has not had test Z performed, the treatment will have to be rescheduled until test Z is performed.

Probabilistic rules, in contrast, are those in which the system estimates the likelihood of a particular outcome based on the data available to it. For instance, a probabilistic rule may indicate that for patients with a certain history of treatment, who are seeking a visit for condition Q, and who have no insurance, there is only a 50% chance they will appear for a visit. Alternatively, a probabilistic rule may indicate that for patients with a particular Aetna insurance plan who have procedure X performed, the mean reimbursement amount is $750, with a standard deviation of $200 and a 10% chance that reimbursement will be denied.

The analytics engine evaluates the corpus of data provided to it by the processing engine and, using appropriate statistical techniques (e.g., machine learning), generates both deterministic and probabilistic rules applicable either generally or to a given patient population, treatment context, or healthcare provider from that data. For instance, the analytics engine analyzes prior outcomes based on the data provided by the processing engine. The analytics engine may also be capable of adjusting dynamically and providing optimized results in response to changing conditions and factors.

The analytics engine, in one embodiment, applies deterministic rules and probabilistic rules to estimate a revenue (or benefit) metric, a cost metric, and a patient probability metric (for instance, the probability the patient will appear for an appointment). These metrics are then combined to generate the visit value score for a given patient visit. In addition, in some embodiments, the analytics engine applies the deterministic and probabilistic rules described above to generate driver metrics. These driver metrics indicate the factors that contributed the most weight to the visit value score (e.g., those in which changes would most impact the score). The driver metrics may be ordered in a ranked list or group. The driver metrics may include prospective changes to factors that would, if realized, most improve the visit value score to the healthcare provider (e.g., confirming that a patient has completed pre-visit lab testing, or changing the healthcare provider for a straightforward visit from a doctor to a nurse practitioner).

The analytics engine may also calculate actual visit values for known prior healthcare appointments. In such cases, the calculation of the visit value may be entirely deterministic, in cases where the cost (in time and material) is known, it is known that the patient did (or did not) appear for the visit, and it is known what payment was received for the visit. Actual visit value scores, and the data used to create them, may be compared with predicted visit value scores for the same or different visits and the results of these comparisons may be used to refine the probabilistic component of the visit value score.

The analytics engine, in another embodiment, applies deterministic rules and probabilistic rules to identify patients who are due for return to the practice for care. “Visit Recall” is the term of art in clinical care for finding patients under the care of a healthcare organization who are due to return for healthcare services. Examples of these services might include annual wellness visits, care for chronic conditions such as diabetes or asthma, or completion of scheduled diagnostic examinations such as pulmonary function tests or laboratory blood tests. In this embodiment, clinical and operational criteria are gathered from retrospective analysis and clinical practice guidelines to establish rules for identifying patients due for return visits. The analytics engine and rules engine work in concert to examine data sets to identify patients eligible for visit recall. Example rules embodiments include patients due for an annual wellness assessment who have not been seen in prior 12 months or a patient due for routine colo-rectal cancer screening who meets age and other eligibility criteria for a screening colonoscopy. In another embodiment of the analytics engine, the system scans for available open visit slots and matches patients with a proposed appropriate appointment. The analytics engine uses deterministic and probabilistic approaches to propose the best match of the patient's needs to the best upcoming appointment opening that maximizes visit value. The analytics engine examines upcoming appointment openings for time of day, day of week, visit lag (i.e., how many days the patient has to wait for the future appointment opening), provider type (e.g., physician versus nurse practitioner), length of appointment opening in minutes, and any diagnostic or therapeutic services openings adjacent to the provider appointment.

In another embodiment, the analytics engine generated a predicted visit value score for the list of patients eligible for visit recall. The patients are organized by visit value score and presented to the clinical care team for review and approval. Approval by the care team triggers communication directly to the patient requesting them to schedule an ambulatory appointment with their provider. Communication with the patient can be done via text messaging, email, telephone, patient portal systems maintained by healthcare providers, postal mail, or other communication modality.

The analytics engine may also allow a healthcare practitioner to analyze patient engagement data. Patient engagement data may comprise any data indicating a patient's interactions with his or her healthcare provider and/or actions that he or she took relating to a particular healthcare visit. For instance, patient engagement data may include data indicating whether a patient checked his or her lab test results online, or whether he or she responded to an email confirming an appointment promptly. The analytics engine may use patient engagement data to determine what types of patient engagement correlate with actual visit value scores being greater (or lower) that expected visit value scores, or higher or lower in an absolute sense. The analytics engine may also use patient engagement data to determine whether certain types of patient engagement (e.g. repeated checks of laboratory results online) lead to increases in provider costs through, for instance, repeated communications to the provider on a particular topic. Further, the analytics engine may use patient engagement data to determine whether certain types of patient engagement with respect to a particular healthcare visit (e.g., again, checking laboratory results online) lead to increased revenue for future visits, by making it more likely that the patient will schedule a follow-up appointment with the healthcare provider.

Further still, the analytics engine may comprise a simulation component that provides, based on the probabilistic and deterministic rules available to the engine, an estimate of the impact that certain changes to the healthcare practice would have on future visit value scores for a certain time period or patient population. For instance, the simulation engine might provide an estimate of the likely change to the visit value scores for a population of patients if the healthcare provider began scheduling appointments earlier in the morning. The simulation engine might also provide an estimate of the changes to visit value scores that would occur if the provider hired a new practitioner with a particular specialty, or hired a doctor instead of a nurse practitioner, or vice versa.

The system may further comprise a scheduling engine that is communicatively connected to the statistics or analytics engine. The scheduling engine utilizes visit value information provided by the analytics engine for actual or prospective patient visits to a healthcare provider and/or to individual healthcare professionals associated with that healthcare provider. The scheduling engine is also communicatively coupled to a source of information regarding the existing schedule of appointments for a healthcare provider and/or individual healthcare professionals associated with that healthcare provider. In some embodiments, the scheduling engine may be communicatively coupled to a computer or server associated with the healthcare provider that maintains appointment scheduling information from that provider.

In other embodiments, scheduling information for a healthcare provider and/or individual healthcare professionals associated with that provider, may be stored in a database or other non-transient data storage structure within or accessible to the scheduling engine, the data integration engine, or the processing engine. In such embodiments, the scheduling information may be retrieved using automated or manual queries to systems associated with the healthcare provider, or may be transmitted by the healthcare provider or its systems using calls to an appropriate application programming interface, as discussed below. In still other embodiments, patient scheduling information may be input directly by personnel associated with the healthcare provider, using a user interface provided by a user interface engine, as discussed further herein.

When presented with information from the statistics or analytics engine regarding a particular patient visit that is to be scheduled, and with existing healthcare provider scheduling information, the scheduling engine may determine what available timeslots the prospective patient visit should be scheduled within to maximize the visit value. The scheduling engine may do this by querying the statistics or analytics engine for information regarding how the visit value might change based on different appointment times and/or associated practitioner availabilities. The scheduling engine may generate an indication of the appointment time and/or practitioner identity that maximizes the visit value score, and may make that information available via an appropriate application programming interface, or by providing it to the statistics or analytics engine.

Alternatively, the statistics or analytics engine may query the scheduling engine to determine what appointment times, and healthcare practitioners, are available, and use the resulting information to identify the slot with the highest visit value score. In either case, the statistics or analytics engine and the scheduling engine interact to identify the appointment time or practitioner to be seen that maximizes a visit value score. For instance, the engines may determine that a particular appointment slot maximizes the visit value because it allows the patient to be seen as soon as possible, although by a nurse practitioner rather than by a doctor. Alternatively, the engines may determine that a particular appointment slot maximizes the visit value because it maximizes the likelihood that a patient will show up for the appointment, or be able to have the necessary pre-appointment tests completed, even if that appointment time is somewhat further in the future.

Although the examples described above illustrate the statistics or analytics engine and the scheduling engine cooperating to maximize the visit value score for a single patient visit, the invention also contemplates that the engines may work to optimize the visit value score for a series of patient visits. For instance, in one embodiment, in determining the optimal appointment time for a prospective patient appointment, the statistics and analytics engine and/or the scheduling engine may take into account the likelihood that a patient with a higher visit value may later seek an appointment for a particular timeslot. This determination may take into account information about prior scheduling patterns for a particular healthcare provider or healthcare practitioner. For instance, the appointment time slot with the highest visit value for a visit by a patient with a flexible schedule and a non-urgent condition may be tomorrow. However, the engines may recommend that this patient should be scheduled for an appointment somewhat further into the future, based on statistical information indicating that the healthcare provider or practitioner is highly likely to receive one or more requests for urgent treatment, which have even higher visit value scores, the day of the open appointment slot.

Further still, the statistics and analytics engine and the scheduling engine may analyze multiple appointment requests simultaneously, and provide outputs indicating the combination of appointment times and/or healthcare practitioners that maximizes the overall visit value score for the combination of appointments. The engines may apply different criteria to determining what combination of visit value scores is preferable to the healthcare provider or practitioners (e.g. maximize the raw sum of visit values, minimize variance among scores, avoid scores below a certain threshold, etc.). Further still, when determining the time slots and/or healthcare providers that would maximize the visit value for a prospective visit or combination of visits, the engines may, in some embodiments, suggest changes to existing appointments that would result in higher overall visit value scores (e.g., would increase the sum of the visit value scores for the healthcare provider or practitioner) or would help the healthcare provider or practitioner meet other defined metrics.

In some embodiments, the functionality of the scheduling engine, as described above, may be included within the statistics or analytics engine, without departing from the invention as disclosed herein.

The system may also consist of API and UI engines that are operatively connected to the analytics engines and can interoperate to provide input and output/analysis functions to users. Specifically, the API engine exposes an application programming interface that allows software resident on other computers or servers to interact with the system of the present invention. For instance, in one embodiment, the API engine may provide application programming interfaces that allows a user, such as a healthcare provider, to access visit value scores, suggested actions to be taken by the patient to increase the visit value score, and/or suggested actions to be taken by the healthcare provider to increase the visit value score. The application programming interfaces provided by the API engine may be in the form of database queries, function or method calls in a particular programming language or communications protocol.

In other embodiments, the API engine can provide a feedback interface that allows a healthcare provider and/or an actual or prospective patient to provide information for communication to the analytics or statistical engine. For instance, the API engine can provide an interface by which a healthcare provider or a patient can indicate that they have taken certain actions that may impact a visit value score, such as confirming an appointment, undergoing necessary pre-visit lab tests or other healthcare related testing, providing insurance or payment information, or changing the time of a visit or the individual healthcare provider (such as a doctor or registered nurse) to be seen. The API engine can provide an interface by which a healthcare provider can request updated visit value scores based on the information provided via such a feedback interface.

In some embodiments, the API engine may provide an interface for the healthcare provider to input information relating to its existing schedule of appointments, or to available appointment times and available practitioners. This information may be general (e.g., Dr. Wang has 30-minute visits available from 8 am to 3 pm on Mondays) and/or specific (e.g., Dr. Smith's 2:30 pm appointment on December 4^{th} is taken). In other embodiments, the API engine may provide an interface for the healthcare provider to request information relating to how the visit values for individual appointments, or for groups of appointments, would vary if certain scheduling changes were made.

The API engine may further provide interfaces through which a healthcare provider may provide, or provide access to, patient engagement data, as discussed above. These interfaces may further allow the healthcare provider to query for information regarding the likely effects of certain types of patient engagement activities (either by the provider or the patient, or both) on the visit value of past or future healthcare visits.

The API engine may further provide a simulation interface that allows a healthcare provider to provide information about potential changes to types of healthcare visit-related activities (e.g. changing available appointment times or the mix of practitioner types, adding or eliminating types of pre-visit reminders, etc.) and receive query results regarding the likely impact of those changes on their future visit values over a particular time scale or patient population.

Further, in one embodiment, the user interface, or UI, engine, provides a user interface, such as a graphical user interface, that presents the output of the analytics or statistical engine to a user. The user interface provided by the UI engine may include a textual or graphical representation of visit value scores for particular scheduled or potential patient visits. For instance, in one embodiment, the interface presented by the UI engine may present a representation of a series of actual or potential patient visits, such as on a calendar, and may use different colors to signify different visit values. For instance, the interface may use one color to signify relatively high visit values, a second color signifying moderate visit values, and a third color signifying lower visit values.

The user interface provided by the UI engine may also include a textual or graphical representation of actions that the healthcare provider can take to improve the visit value score, or of actions that might lower the visit value score. For instance, the UI engine may note that a particular patient's visit value may improve if he or she is provided with information on transportation to the healthcare facility, or if he or she is scheduled to see a nurse practitioner instead of a doctor. In one embodiment, the user interface may include an indication of the relative ordering of the degree to which such suggested actions will improve or affect the visit value score. In some embodiments, this user interface is interactive and dynamically updatable, such that the healthcare provider can indicate that they have taken certain suggested actions presented by the UI engine, and can, in some embodiments, see an updated representation of the visit value score that accounts for those actions.

In further embodiments, the user interface provided by the UI engine may include a textual or graphical interface that is accessible to patients who have an actual or prospective visit scheduled with a healthcare provider. This interface may provide the actual or potential patient with information about the visit, such as its time and location, and transportation and payment options. The interface may also include reminders for the patient that are pertinent to the upcoming visit, including reminders regarding information or records to bring to the appointment or tests or other actions that need to be completed or performed prior to the appointment. In some embodiments, the interface presented to the patient is interactive and dynamically updatable, such that the patient can, for instance, confirm an appointment or indicate that he or she has completed necessary pre-appointment tests. In some embodiments, the interface presented to the user may be retrieved and controlled directly from the UI engine. In such embodiments, the healthcare provider may be informed, for instance through its own interface to the UI engine, that a patient has taken certain suggested actions and that his or her visit value has therefore changed. In other embodiments, the interface presented to the user may be controlled by a server or other computer located at or controlled by the healthcare provider, which retrieves information from the UI engine and prepares it for presentation to the user.

In some embodiments, the user interfaces provided by the UI engine may comprise one or more scheduling interfaces. These scheduling interfaces may allow the healthcare provider, or its practitioners, to input and track information regarding available appointment slots for healthcare practitioners, as well as information regarding appointment slots that have been taken (including information regarding the patient taking the appointment, information about the nature of the visit and necessary prerequisites, insurance or other financial information pertinent to the appointment, and the like). The scheduling interfaces may also serve to provide the healthcare provider or practitioner with scheduling recommendations that reflect information from the statistics or analytics engine and/or the scheduling engine as to how to schedule one or more appointments to maximize their individual or overall visit value score(s).

The UI engine may also comprise interfaces to allow a healthcare provider to input patient engagement data or to review information from the analytics engine on the likely impact of particular types of patient engagement on visit value scores. The UI engine may also comprise interfaces that allow a healthcare provider to set up and review the results of simulations run by the analytics engine that evaluate the likely or potential impact of certain changes to healthcare-related activities on future visit value scores.

Yet another embodiment of the present invention may be described as a system comprising a patient data set stored in a patient database, the patient data set including at least a patient identifier, an appointment date, an appointment type, and required appointment prerequisites; a healthcare provider data set stored in a healthcare provider database, the healthcare provider data set including a plurality of physician types associated with the healthcare providers of the healthcare practice, a plurality of appointment options for each physician type, and revenue of a plurality of healthcare visits; a financial data set stored in a financial database, the financial data set including a plurality of visit costs, a plurality of payment methods, a plurality of insurance types, and a plurality of payment outcomes; a controller in communication with each of the patient database, the healthcare provider database, and the financial database; and a memory coupled to the controller, wherein the memory is configured to store program instructions executable by the controller such that, in response to executing the program instructions, the controller: calculates a visit value score associated with a scheduled future healthcare visit for a patient end user, wherein the visit value score is based on a visit input data set comprising at least one data element from each of the patient data set, the healthcare provider data set, and the financial dataset; wherein, when the calculated visit value score is below a pre-defined score limit, the controller: identifies one or more of the data elements in the visit input date set that may be altered by an action to be taken by the patient end user or a healthcare provider end user to improve the calculated visit value score; and notifies either the patient end user or the healthcare provider end user of the action to be taken with instructions to take the action.

The system above may also, in response to executing the program instructions, the controller further: receives notice that at least one data element in the visit input data set has been revised; calculates a revised visit value score associated with the scheduled future healthcare visit for the patient end user based on a revised visit input data set; and wherein, when the calculated revised visit value score is below the pre-defined score limit, the controller: identifies one or more of the data elements in the revised visit input date set that may be altered by an action to be taken by the patient end user or a healthcare provider end user to improve the calculated visit value score; and notifies either the patient end user or the healthcare provider end user of the action to be taken with instructions to take the action.

The visit value calculated may be displayed upon a graphical user interface or used to generate a report. The notification generated by this system may be an email, test message, or SMS message. At least one data element from the patient data set, the healthcare provider data set, or the financial dataset may be supplied from an external data source. The action to be taken with instructions to take the action may also be displayed upon a graphical user interface or sent as an email, test message, or SMS message.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a diagram of an embodiment of a healthcare visit value calculation system.

FIG. 2 is a flowchart illustrating how data is analyzed by the healthcare visit value calculation system.

FIG. 3 is a flowchart of how the visit value calculation system accepts data concerning a new patient.

FIG. 4A is a patient welcome screen of the healthcare visit value calculation system's GUI.

FIG. 4B is a healthcare team summary screen of the healthcare visit value calculation system's GUI.

FIG. 5 is a patient communication screen of the healthcare visit value calculation system's GUI.

FIG. 6 is a healthcare provider calendar screen of the healthcare visit value calculation system's GUI.

FIG. 7 is a healthcare provider reports screen of the healthcare visit value calculation system's GUI.

FIG. 8 is a healthcare provider daily schedule screen of the healthcare visit value calculation system's GUI.

FIG. 9A is a patient communication screen of the healthcare visit value calculation system's GUI.

FIG. 9B is a patient communication screen of the healthcare visit value calculation system's GUI after a patient's first visit.

FIG. 10 shows a screen displaying patient labs and tests ordered, from the healthcare visit value calculation system's GUI.

FIG. 11 is a diagram of data sources for an embodiment of the healthcare visit value calculation system's GUI.

FIG. 12 is a flowchart which demonstrates how a data integration between external data sources and the centralized private server network operates for an embodiment of the healthcare visit value calculation system.

### DETAILED DESCRIPTION OF THE INVENTION

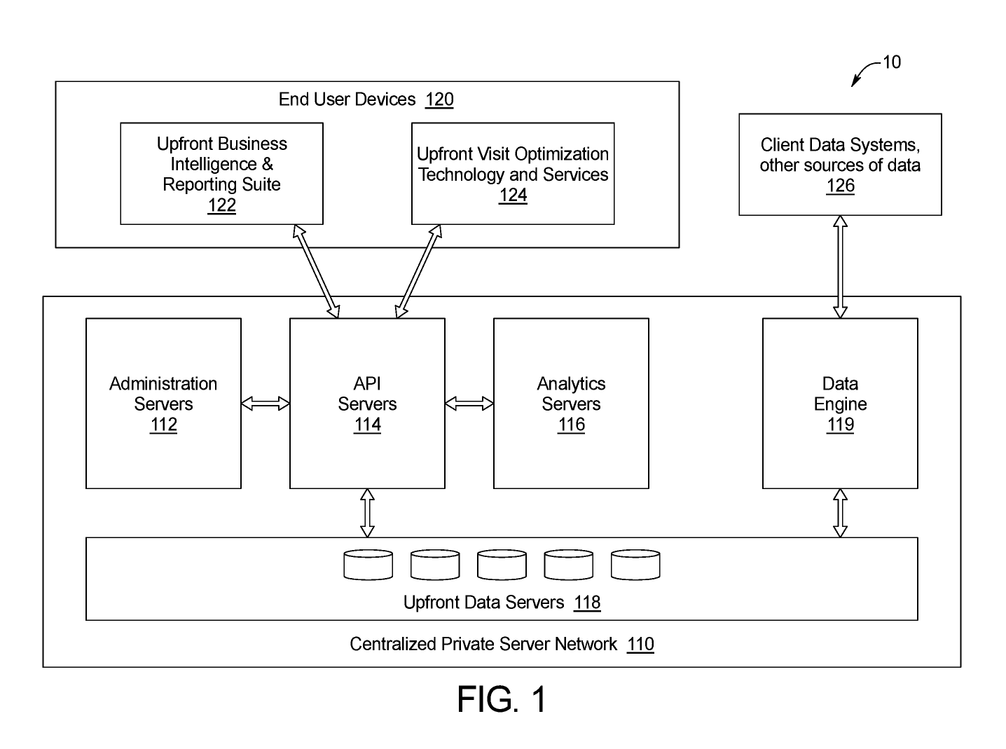

FIG. 1 is a diagram of an embodiment of a healthcare visit value calculation system 10. As shown in FIG. 1, in one embodiment, the visit value calculation system 10 may feature a centralized network of servers 110 (with processors, memory, and a networking interface), statistical software, healthcare database(s), and end user devices 120. The end user devices 120 may be desktop computers, laptops, tablets, ‘land line’ telephones, FAX machines, SMS, smartphones; or mobile devices. In the embodiment shown, the centralized server network 100 communicates with various healthcare databases and end user devices 120 to obtain information concerning various data points which impact the value of a given visit of a patient to a healthcare provider. The data points may include variables pertaining to: a patient's options to come earlier and see alternative clinicians other than a physician, computerized telehealth appointment instead of in-person visit, a patient's need to have a test completed before a visit, the likelihood to show up to the visit, a patient's likelihood to meet their deductible, time of day & day of week of appointment, the costs of providing the appointment, and quality care gaps for that patient (e.g., routine diabetic screenings due). Other information collected by the system may include insurance contractual terms for: payments for quality of care provided, patient satisfaction, and reimbursement of services provided.

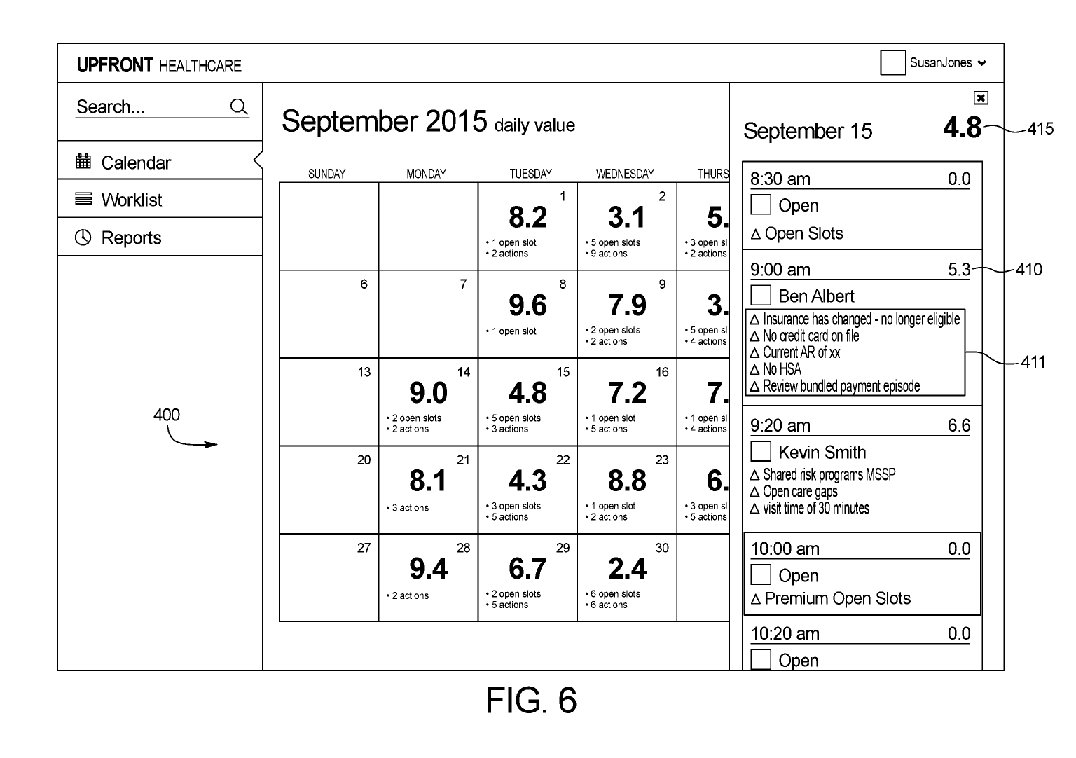

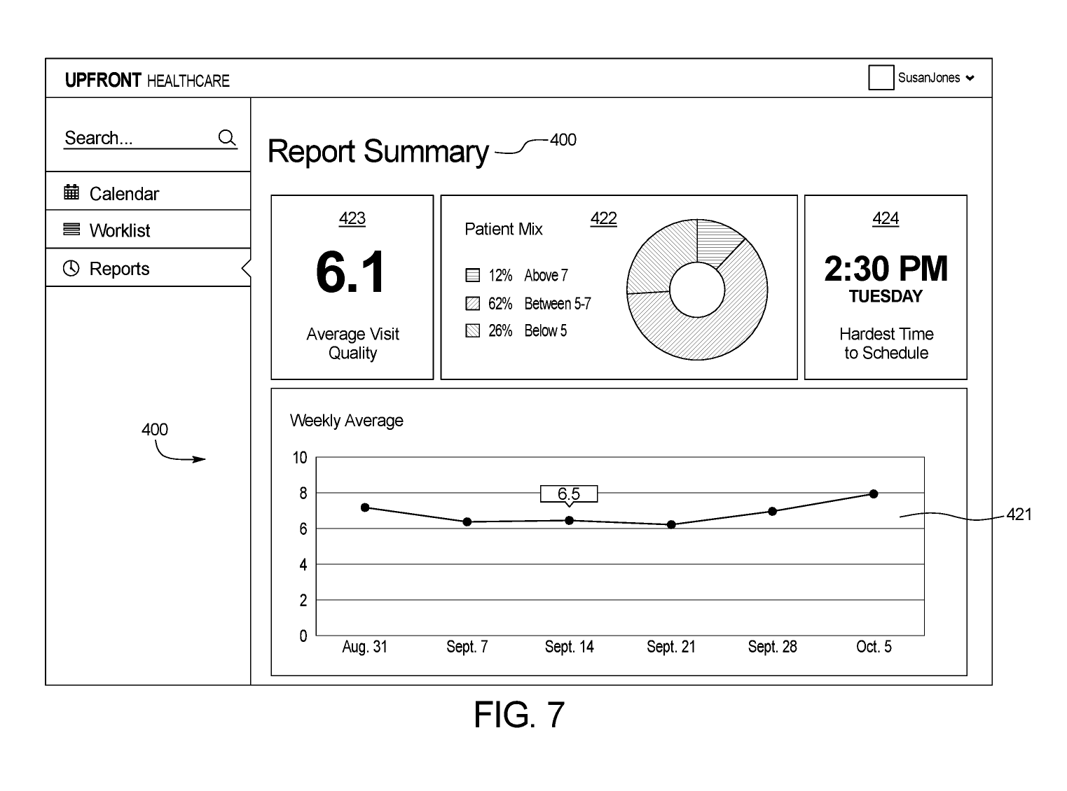

Once these various data points are transmitted to the centralized network of servers 110, the visit value calculation system 10 will then apply various statistical models to the collected data, creating a new set of analyzed data points. These analyzed data points are used to calculate a visit value (illustrated in FIG. 6). The visit value score and the statistical data generated along with the score are also evaluated by various probabilistic and deterministic rule sets. These rule sets trigger various system alerts and notifications sent to designated end users. One example of a rule: an upcoming appointment with a physician would have substantially higher visit value if completed with a nurse practitioner instead. The analyzed data points and actions are also fed into an end user reporting interface (illustrated in FIG. 7) for clinicians, practice managers, access center employees, healthcare executives, data analysts, et al. to review.

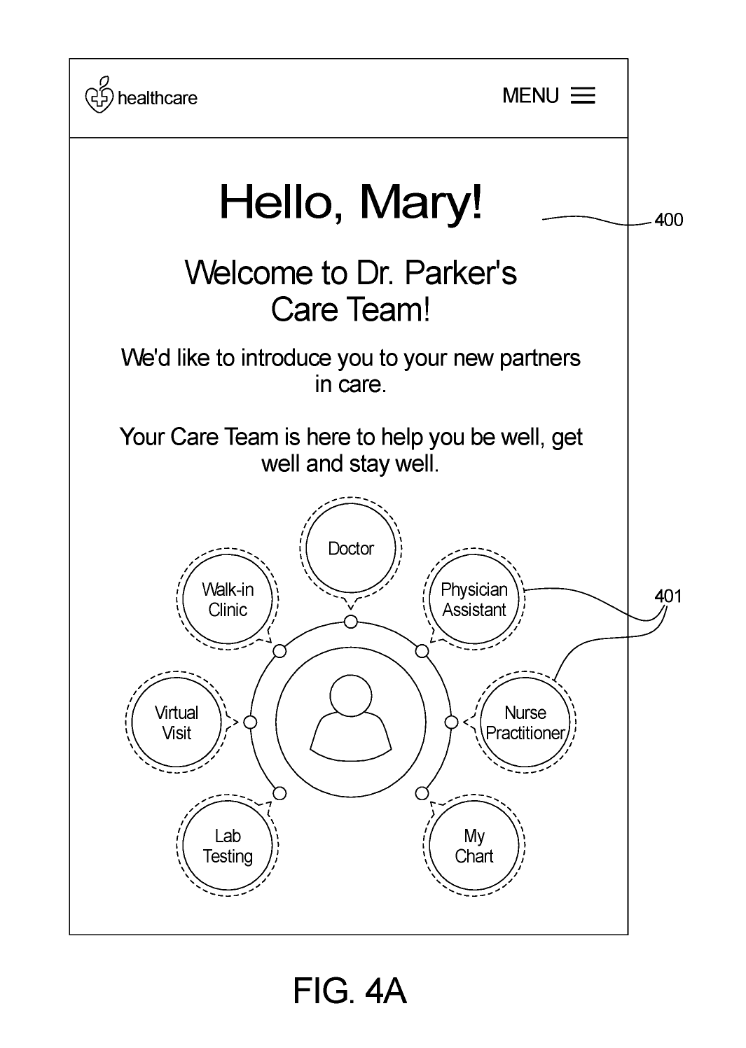

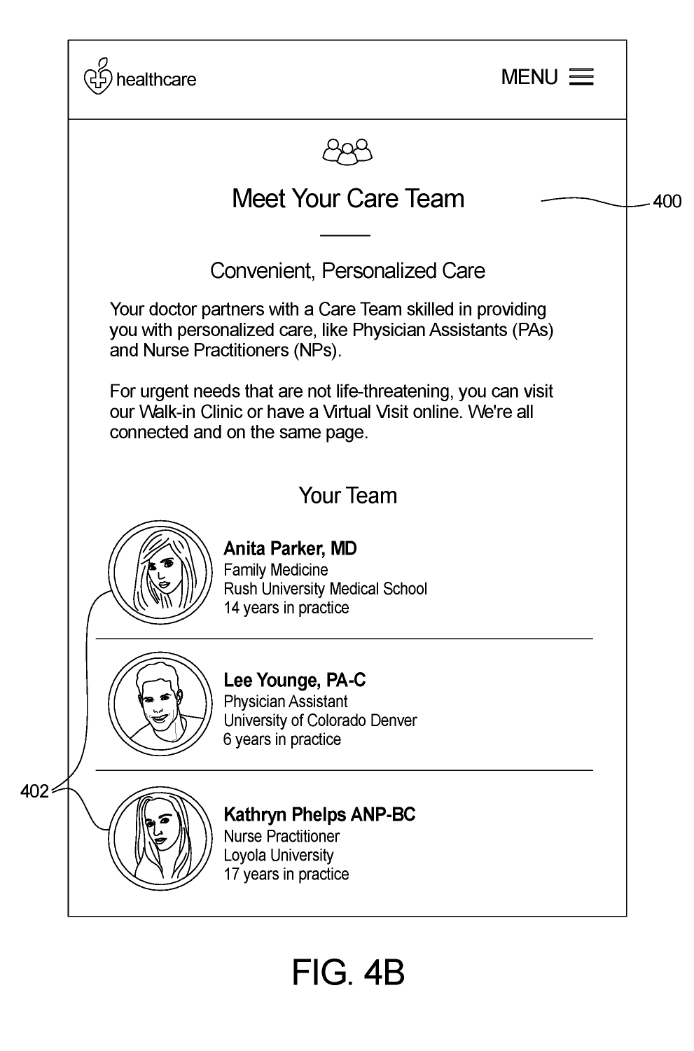

Also shown in FIG. 1, the end user devices 120 may run various healthcare software solutions. Once such software solution is the Upfront Healthcare business intelligence and reporting suite 122. This software solution may be a stand alone application, series of applications, or hosted software solution (e.g., cloud hosted, software as a service (SaaS) hosted, etc.). The Upfront Healthcare business intelligence and reporting suite 122 may act as the point of contact for most end users, with a graphical user interface 400 (GUI, see FIG. 4A-FIG. 10) comprised of various screens being displayed to various types of end user (e.g., patients, doctors, administrators, etc.).

The end user devices 120 of this embodiment of the system 10 may also feature a software application or applications which provide healthcare visit optimization services. The Upfront healthcare visit optimization services software 124 may be integreated with the business intelligence and reporting software suite 122 and accessible from the same GUI 400 or exist as stand alone application(s) depending on the ideal implamentation of a given embodiment of the system 10. In the diagram shown, the visit optimization services software 124 and business intelligence and reporting software suite 122 both send and receive data from the centralized private sever network 110. The end user devices 120 utilizing this embodiment of the system 10 may also send and receive data from the centralized private sever network 110 via other healthcare data system (external sources of data 126) including integrations with other healthcare data systems and sources including patient reporting systems, insurance databases, etc. The external sources of data 126 may also be automated data integrations which send and receive data from the centralized private server 110 without the need for end user intervention.

The centralized private server network 110 may be composed of administration servers 112, application program interface (API) servers 114, analytics servers 116, data servers 118, and a data engine 119. In this embodiment, the administration servers 112 handle tasks including rules configuration and system administration as well as client dashboard maintenance. The administration servers 112 may communicate with the API servers 114, Analytics servers 116, and Data servers 118. It should be noted the servers shown here may be physically separate pieces of hardware or virtually separate and hosted upon a single physical server, cloud based server solution, etc.

The application program interface (API) servers 114 may act as the reception point of end user inputs and monitor end user actions, tasks, and notifications. The analytics server 116 in this embodiment is dedicated to healthcare data analytics, rules processing, action creation, and action aggregation. The admin, API, and analytics servers 112, 114, 116 all communicate with the data servers 118 which may store all data related to system 10 usage, healthcare visit information, past healthcare records, and any other data which is useful to one of the system's 10 functions.

The data engine 119 acts to send and receive data from various sources including other healthcare data systems as well as external data sources 126. The data engine 119 handles staging data, mapping data, and batching data in addition to sending real time data and processing incoming data. The external data sources 126 may also include data from other instances of the healthcare visit value calculation system 10.

It should be noted that the centralized private sever network 110 might be a Microsoft Azure Network or another server network capable of similar functionality. The data stored in the internal databases 118 and acquired from external data sources 126 may be described as data sets, records, data files, etc.

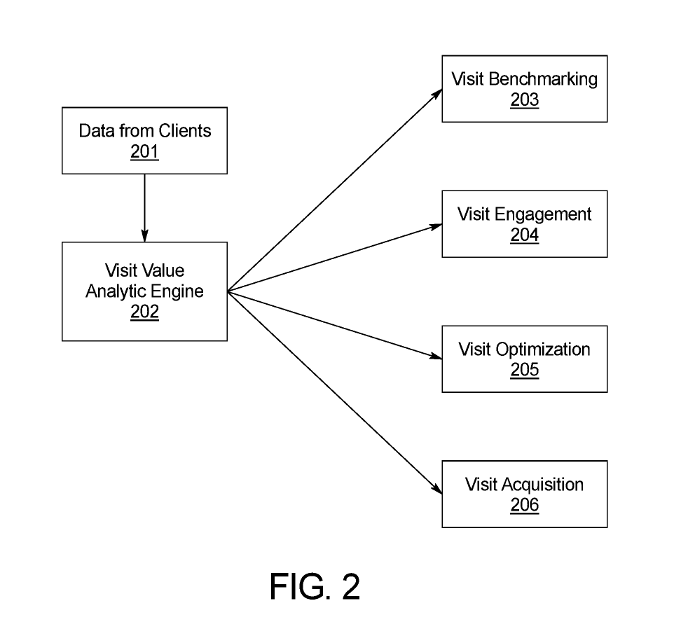

FIG. 2 is a flowchart illustrating how data is analyzed by the healthcare visit value calculation system 10. As shown in FIG. 2, the system begins with various data points concerning a visit (e.g., insurance terms, contractual terms, patient information, doctor information, scheduling information, etc.) at step 201 and then feeds this data into the centralized private server network 110 (specifically the analytics server 116) (step 202). This analytics server 116 and its hosted analytics engine(s) may utilize data not only concerning a given doctor and patient, but may also use aggregated data from various external sources (e.g., nationwide healthcare outcome reports). The analytics engine produces new data set(s) using these internal and external data sources. This newly analyzed data set may include a visit value or visit value(s) which may be used on their own, or in combination with other data to trigger alerts and notifications to be sent out by the system 10. Such alerts and notifications are created automatically in this embodiment by a rules engine, which is a module of the system that examines the analyzed data set(s) via various probabilistic statistical and deterministic approaches. An example of a probabilistic approach is predicting the impact of providing transportation on a patient's likelihood to attend their appointment instead of missing it. An example of deterministic approach rules: if the patient is due for a procedural screening examination (e.g., colonoscopy or mammography), provide education to the patient.

Data from the centralized private server network's 110 analytics engine, rules engine, etc. may then be used by the system 10 for various tasks. One such task is generating visit intelligence (a play off the term business intelligence) which enables healthcare providers to examine the value of a given visit via graphs, reports, and other analytic tools (see FIGS. 6-7). This step 203 is also known as visit benchmarking as the data produced by the system 10 is primarily used by healthcare executives, data analysts, and other healthcare leadership positions to help identify opportunities to improve healthcare value.

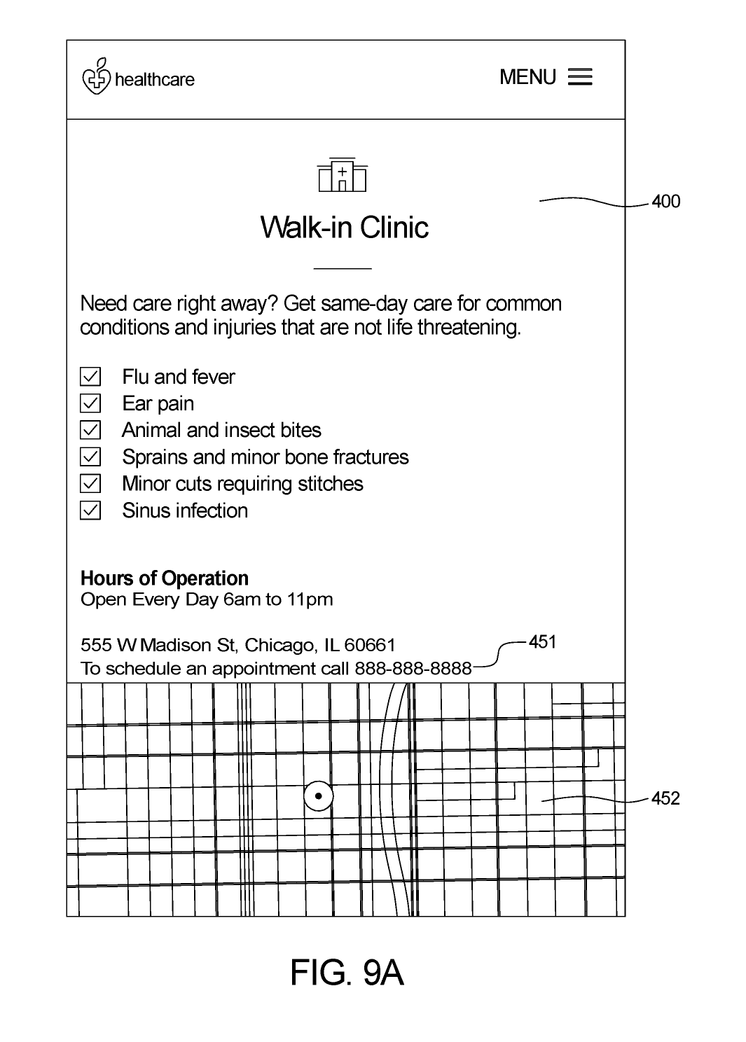

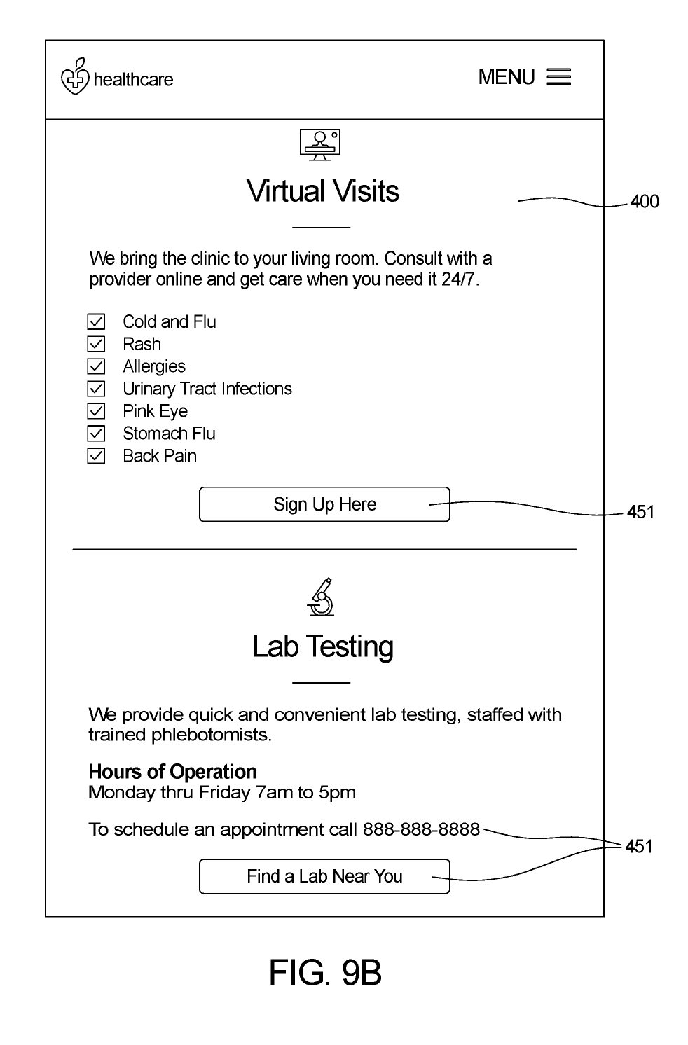

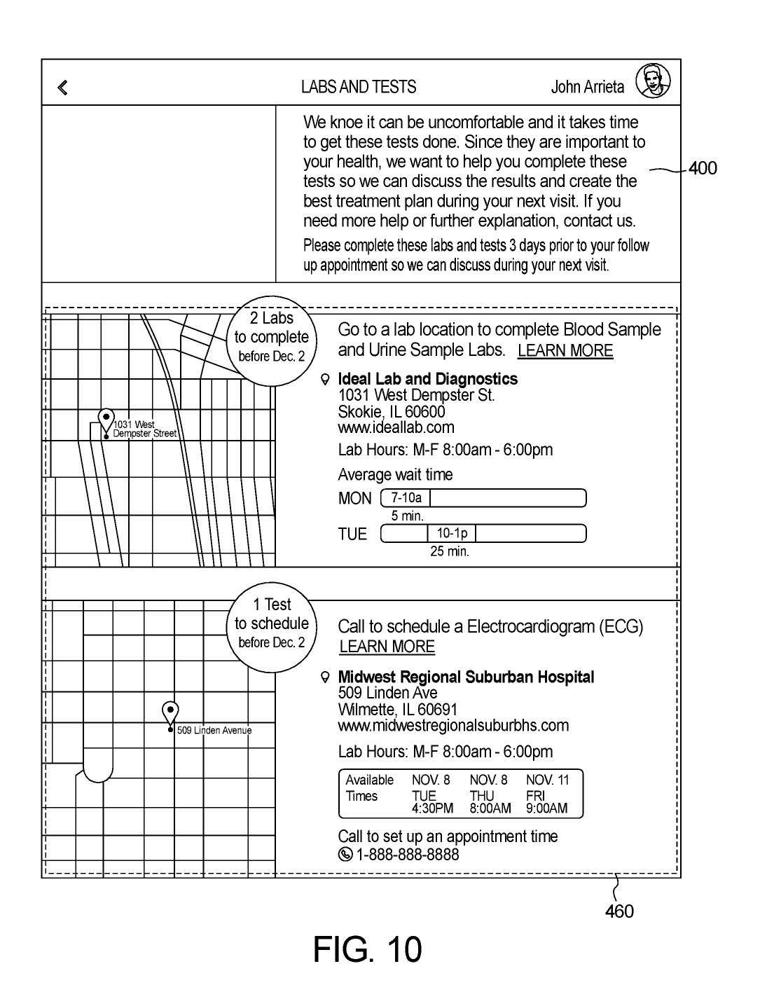

Another task the system may carry out is visit engagement (step 204) in the form of direct-to-patient communication with goal of completing pre-visit tasks to optimize the actual visit (see FIGS. 9-10). This direct-to-patient communication may include patient education on various subjects relevant to a given visit (e.g., what a certain specialist can and cannot do), offering scheduling alternatives (e.g., telehealth or schedule with a Nurse Practitioner), and also ensure pre-visit testing occurs by sending reminder notifications to patients.

Yet another task the system may assist in is the task of visit optimization (step 205) by using the data from the analytics engine and rules engine (hosted upon the centralized private sever network 110) to queue tasks for upcoming visits and prioritize them to improve visit value score. For example, the system 10 may send notifications to health care providers to obtain insurance information from the patient or perform a blood test to ensure the visit is as efficient and valuable as possible.

Finally, the system 10 can also aid in visit acquisition (step 206). Visit acquisition may require scheduling pateints for routine checkups, etc. with the system 10 capable of monitoring a healthcare practice or organization's patients to ensure any follow up appointments, routine check-ups, etc. are scheduled. This scheduling may be done automatically by the system 10 or by the system 10 generating notifications and alerts for healthcare staff and patients to schedule such meetings.

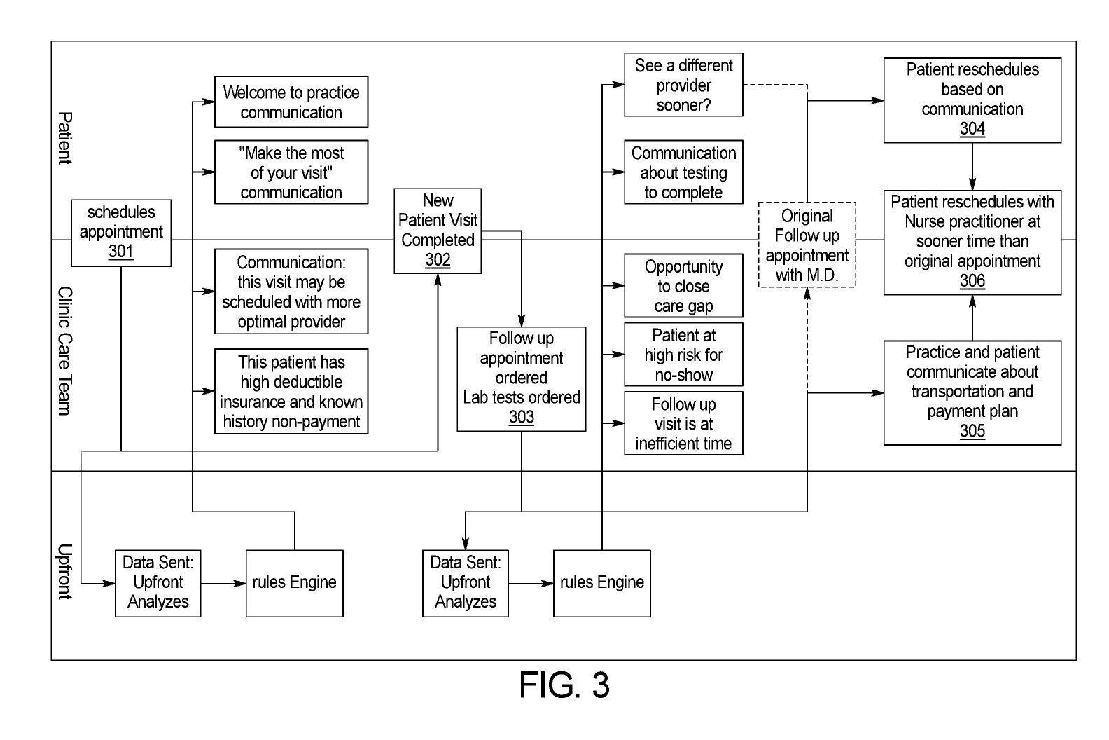

FIG. 3 is a flowchart of how the visit value calculation system 10 accepts data concerning a new patient. As shown in FIG. 3, the system's 10 various functions begin with step 301 of scheduling an appointment. Appointment scheduling data in this embodiment is transmitted to the system 10 automatically from other healthcare management software (e.g., external sources of data 126). Once an appointment is scheduled, the system 10 collects, collates, and analyzes various data points (discussed in FIGS. 1-2) and passes this information on to the rules engine (also discussed in FIGS. 1-2). At this point, the rules engine will trigger the system 10 to display various notifications and alerts to the patient and healthcare provider. These alerts and notifications may be displayed via a graphical user interface 400 (see FIGS. 4-10), text message, SMS message, email, automated telephone call, etc.

In this example, upon scheduling a first appointment, the patient is shown notifications (in the form of system GUI 400 screens) which welcome them to the healthcare providers' practice and then also advises them how to make the most out of their visit (e.g., what tests to get done ahead of time, etc.). When a first appointment is scheduled, GUI 400 screens are presented to the healthcare provider and other clinical and operational staff which display notifications. Notifications can also be provided by other communication modalities, such as text message, smartphone applications, or FAX machines. In this case, the notifications tell the provider that the incoming patient may be scheduled with a more optimal provider and that the patient has a high deductible and history of non-payment. These are examples of information relevant to the potential value of the upcoming visit or lack thereof.

Once the appointment is registered as completed (step 302) by the system 10, information regarding the visit is recorded by the system 10 including, in this example; that a follow up appointment is scheduled along with lab tests (step 303). Information regarding the appointment as well as the follow up steps ordered are fed into analytics engine along with other healthcare data (as discussed in FIGS. 1-2) for analysis and then on to the rules engine to trigger various notifications. It should be noted that the various analytics tool(s) and rules engine(s) are hosted by the centralized private server network 110 and its various virtual and or physical servers which carry out various tasks (e.g., data storage upon the data storage server 118 and data analytics upon the analytics server 116, etc.).

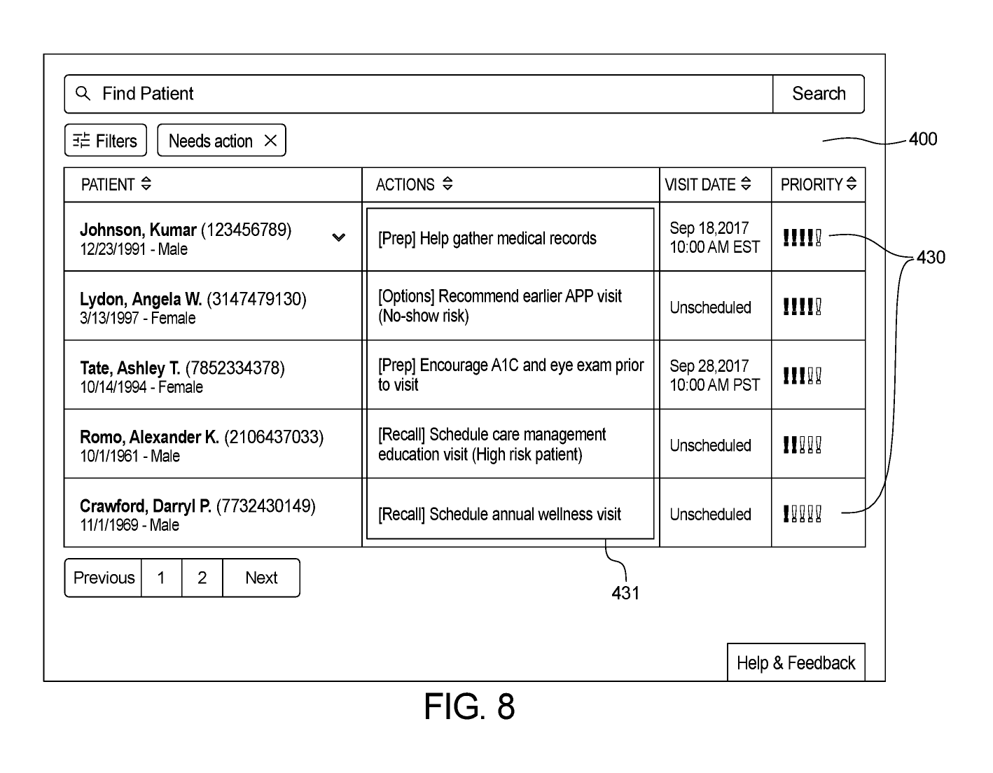

In this example, the rules engine displays notifications to the patient to reschedule with an alternative to a physician visit, which would be predicted to increase the visit value of the encounter for both the patient and healthcare provider. This value would increase, for example, due to using a lower cost clinician and a patient having the encounter sooner and being more likely to attend the appointment. If the patient completes the tests ordered by the doctor, the system 10 will keep track of this information as well and also let all parties know which tests remain uncompleted (see FIG. 10) via a GUI 400 screen or other notification. As for the healthcare provider, they are provided with suggestions about how to improve the visit value for the given patient (see FIG. 8) via another GUI 400 screen and via notifications such as an alert regarding the patient's risk of no-showing and a suggestion to move the next appointment to a more optimal time.

Finally, progressing through the flow chart in FIG. 3, the patient opts to take the advice of the system and reschedules their visit with a healthcare practitioner who can better address their needs (step 304). The system 10 tracks this rescheduling, providing reminders to the patient and healthcare providers about concerns surrounding the new visit (e.g., transport information, payment information at the new clinic, etc.) (step 305). The system 10 then finally records this rescheduled appointment information and recalculates the visit value forecast (step 306).

FIG. 4A is a patient welcome screen of the healthcare visit value calculation system's 10 GUI 400. As shown in FIG. 4, when a new or existing patient wishes to schedule an appointment with a given healthcare provider, they may navigate to a welcome screen which lists all healthcare providers at a practice. The system 10 provides education on the value of physician alternatives, such as telemedicine or nurse practitioners. The system's 10 rules engine determines opportunities to encourage use of those provider alternatives when it predicts it will raise the overall visit value score (see FIG. 6). Once a patient expresses interest in scheduling or changing an appointment via the GUI 400 screen's buttons 401, the system 10 provides communication options for scheduling/rescheduling the visit.

Once the appointment is scheduled/rescheduled, the system 10 begins collecting data to use in its visit value analysis. This data may be pulled from any number of internal (e.g., data storage servers 118) and external (e.g., external data sources 126) resources including electronic medical records, insurance company records, and also data manually input into the system 10 concerning the patient.

FIG. 4B is a healthcare team summary screen of the healthcare visit value calculation system's 10 GUI 400. A shown in FIG. 4B, a short biography 402 of each healthcare team member currently assigned to a patient may be displayed to patient's upon their end user devices 120. This provides increased transparency to patients and prevents any confusion on who they will be seeing for a given appointment.

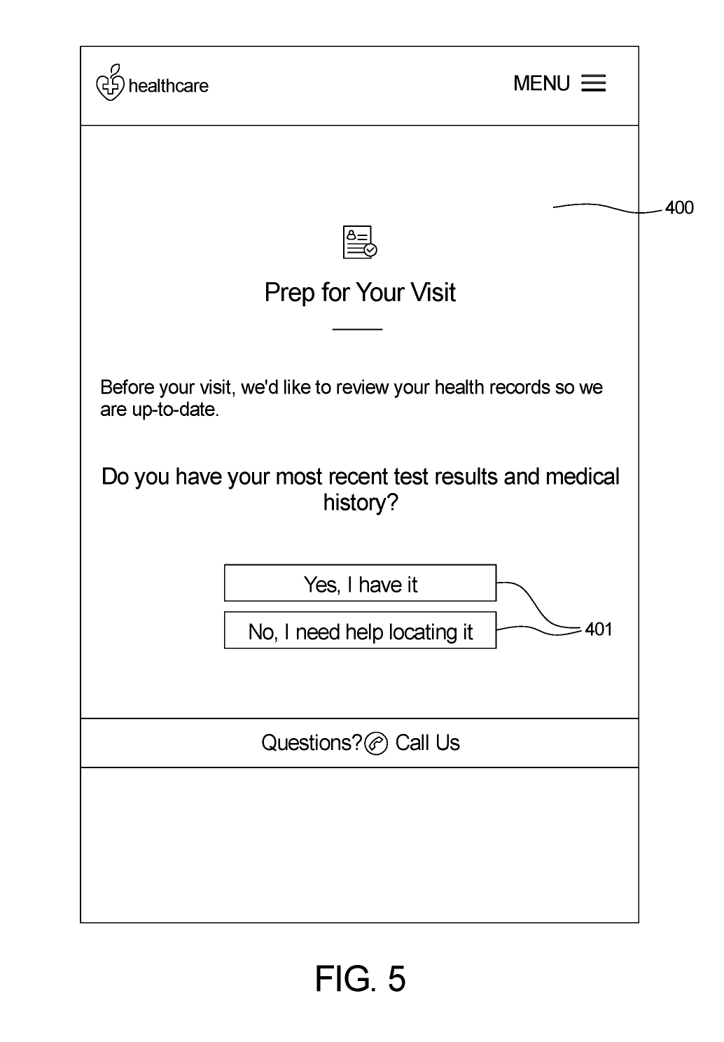

FIG. 5 is a patient communication screen of the healthcare visit value calculation system's GUI 400. In this example, the patient is sent a text message which directs them to a website via hyperlink, etc. This website displays a portion, or all of the system's 10 GUI 400 which enables pateints to interact with the system 10 to determine if they have submitted all their medical records and recent test results for an upcoming patient visit. Entrance of such information is enabled by buttons 401 which feature standardized responses. The GUI 400 may also collected information from pateints via free response fields, drop down menus, radio buttons, etc. In another embodiment, this website may offer different appointment times and dates or offer an appointment with a different type of provider such as a nurse practitioner instead of physician. Depending on the patient responses upon the GUI 400, they may be sent additional information, contacted via telephone, or asked to contact the clinic at their convenience.

It should be noted that the system's 10 GUI 400 may be accessed via any type of end user device 120 capable of displaying a web page and or stand alone application including desktop computers, laptops, smartphones, tablets, smart watches, smart home devices, smart car displays, etc.

FIG. 6 is a healthcare provider calendar screen of the healthcare visit value calculation system's 10 GUI 400. As shown in FIG. 6, once the system 10 has analyzed upcoming patient visits for a given healthcare provider, the system 10 will generate many different analytical pieces of data which can be used to optimize the value of visits by date range, clinic, or individual visit. For instance, in this example, the date September 15 has a mediocre value of 4.8 (out of 10) assigned for the day indicated by the daily visit value indicator 415. This score is calculated based off an average of the visit values 410 for that day, with open slots being given a value of 0.0 as they offer no value to the healthcare provider.

In addition to accounting for open slots, the system 10 also evaluates each visit and gives it a score 410 from 0-10. In this case, one patient's score 410 is 5.3, hampered by the fact that they have no insurance or credit card on file and prior missed appointments. Another patient has a score 410 of 6.6 and has visit value factors 411 identified as negative by the system 10 related to expected services provided during the visit and gaps in their care history. These data points are just a few examples of the numerous visit value factors 411 the system 10 can account for when calculating a visit's value.

The system 10 can also monitor various datapoints related a given visit value factor 411 in real-time (or near-real time) so if the patient with a score 410 of 5.3 calls in and provides their new insurance information, their visit score 410 may be boosted. The system 10 also enables end-users to sort these factors 411 by various criteria; e.g., maximal impact on visit value score, insurance type, or patient characteristics such as age, distance from clinic, or likelihood to no-show. This sorting functionality enables prioritization of actions to improve the visit value in advance of the visit, using real-time data.

FIG. 7 is a healthcare provider reports screen of the healthcare visit value calculation system's 10 GUI 400. As shown in FIG. 7, the system may generate various reports, charts, and graphs which demonstrate the value of a given healthcare provider's day, week, month, year, etc. In this example, a bi-monthly report 420 is shown in the form of a line graph 421, with a pie chart 422 demonstrating a breakdown of patient values in the time period. The average visit value score for the given healthcare provider 423 is also shown in addition to the system's determined “hardest time to schedule” for the week 424. All of the data shown may be updated in real time, with the reporting screen being customizable to data set(s) and reports each end user finds most helpful.

FIG. 8 is a healthcare provider daily schedule screen of the healthcare visit value calculation system's 10 GUI 400. As shown in FIG. 7, the system can generate a daily schedule 430 which lists all the patients a healthcare provider is set to see on a given day. The list also includes ranked optimization activities 431 (that correspond to the visit value factors 411 shown in FIG. 6) which may be carried out to maximize the potential visit values for upcoming appointments. For instance, a patient named John Arrieta is scheduled at 11:15 AM on the schedule shown. If the healthcare provider gathers this patient's healthcare history during the visit (or before), the value of the visit will be bumped up by 2 points, which is significant given the scores are calculated from 0-10 (see FIG. 6).

FIG. 9A is a patient communication screen of the healthcare visit value calculation system's 10 GUI 400. As shown in FIG. 9A, patients may access the system 10 via a website (or mobile device application, etc.) and be shown a screen which lists upcoming (and previous) appointments which are scheduled or should be. For instance, the patient in this example (the same one discussed in FIG. 3) had scheduled a first visit with the healthcare provider on Nov. 1, 2016. Since this patient is new, to maximize the value of this visit, the system displays a clickable link 451 for the patient to communicate with appropriate personnel to provide relevant information such as prior medical records, insurance information, credit card info, transportation needs, etc. The screen shown in FIG. 9A also features a map 452 which enables the end user to identify where the walk-in clinic (in this example) is located.

FIG. 9B is a patient communication screen of the healthcare visit value calculation system's 10 GUI 400 after a patient's first visit. As shown in FIG. 9B, after the first visit was completed for the patient discussed in FIGS. 3 and 9A, the rules engine (see FIG. 1-2) detected a predicted low visit value for the patient and so the system 10 suggests a teledermatology appointment as an alternative to an in-office visit. In this example, the teledermatology appointment provides a better value proposition to the present healthcare provider given the patient's insurance coverage. The patient derives this added value from being seen sooner and having a lower cost of care. Accordingly, the GUI 400 displays to the patient user a clickable link 451 for them to communicate with appropriate personnel to schedule a teledermetology visit. The link 451 may also display a pop-up window or separate webpage which enables patient end users to view a schedule of currently available times for their visit so the patient user can directly schedule a time for their visit.

Additionally, in this example, a doctor user has also scheduled lab tests after the first appointment which show up on the patient communication screen and in the patient's appointment list 460 (see FIG. 10). The system 10 informs the patients to complete the tests ahead of their upcoming appointment and provides clickable links 451 to enable patients to quickly schedule or complete the appointments. Completing tests ahead of time helps increase the value of the future visit. and avoid repetitive, low value visits due to incomplete diagnostic testing. The system 10 will also inform the patient there are appointments with other healthcare providers to help steer them towards a provider who better suits their needs.

FIG. 10 shows a screen displaying patient labs and tests ordered, from the healthcare visit value calculation system's 10 GUI 400. As shown in FIG. 10, in the hopes of preventing “leakage” (e.g., patients going outside a given healthcare provider to get tests done or scheduling a follow up appointment with another practice), the system 10 will provide patients with an appointment list 460 featuring maps, current wait times, and contact information for labs and clinicians associated with the given healthcare provider to keep the patients from going outside of the healthcare provider's revenue stream.

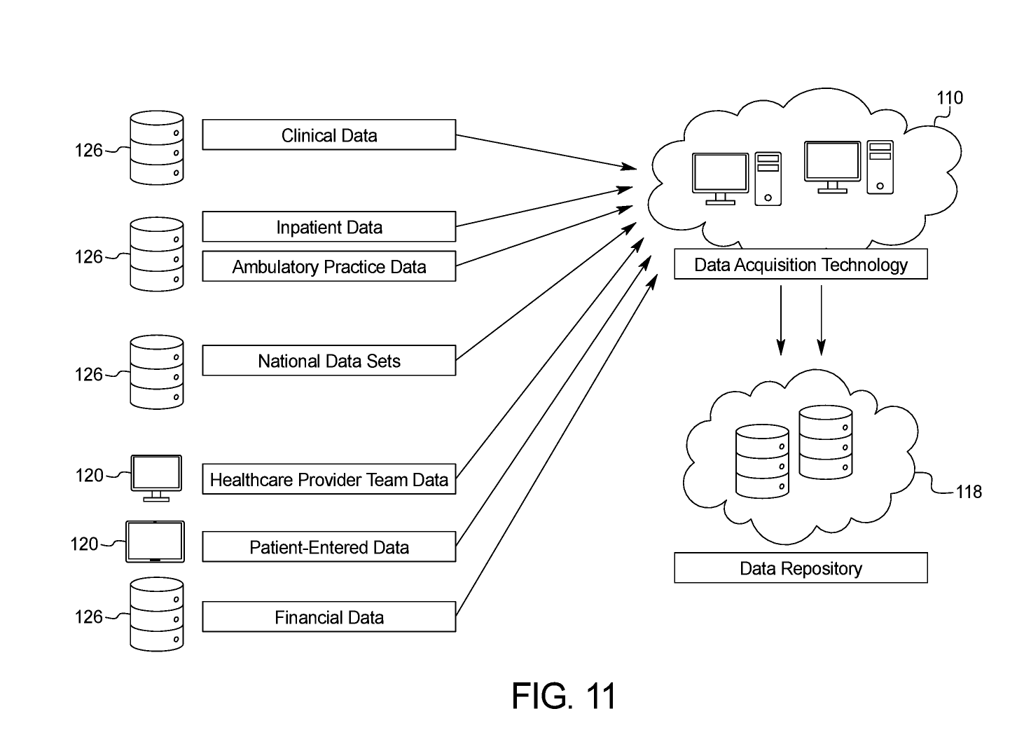

FIG. 11 is a diagram of data sources for an embodiment of the healthcare visit value calculation system's 10 GUI 400. As shown in FIG. 11, the system 10 may collect data from external data sources 126 as well as end user devices 120. All of this information is then collated and stored by the system 10 automatically within the system's 10 internal data storage servers 118. For example, if the system 10 has stored a record stored within one of its various databases (in turn stored upon the internal data storage servers 118) the system 10 can automatically update this record has various health careprovider end users and the patient themselves update information about their care. The system 10 can also reach out to, for example, and insurance providers coverage database (en external database 126) to determine if various treatments and appointments designated for a given patient will be covered. All of this data is obtained by various data acquisition technologies hosted by the system's 10 centralized private server network 110 including administration servers 112, application program interface (API) servers 114, analytics servers 116, data servers 118, data engine(s) 119, etc. (see FIG. 1).

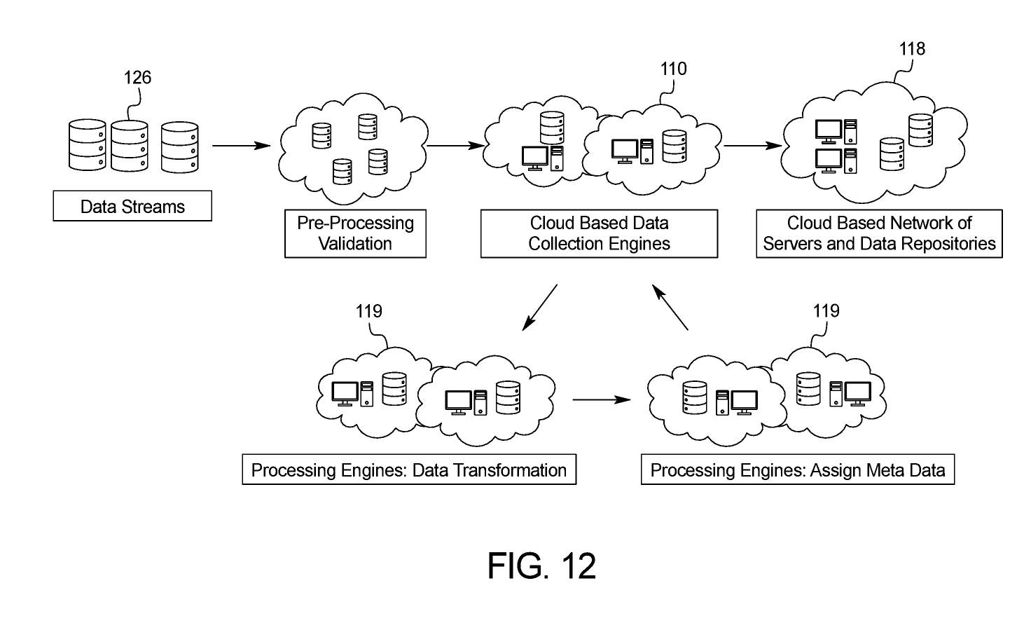

FIG. 12 is a flowchart which demonstrates how a data integration between external data sources 126 and the centralized private server network 110 operates for an embodiment of the healthcare visit value calculation system 10. As shown in this example, various data streams from external data sources 126 may, at a first step undergo pre-processing validation. Such validation may occur externally to the system's 10 centralized private server network 110 with the server hosting the external data source 126 conducting the validation. Once the data is validated, it may then be transmitted via the internet or another means of data communication to one or more cloud based data collection engines (e.g., data engine 119, see FIG. 1). After the data is acquired by the data engine 119 it is then stored within the centralized private server network 110, specifically the data storage server(s) 118. In this example, the centralized private server network 110 and corresponding data storage servers 118 are cloud based.

The data engine 119 discussed above may carry out various tasks when acquiring data including transforming the acquired data to a format which is consistent with other data stored within the internal data storage server(s) 118. This transformed data may also have various pieces of metadata assigned which enable various additional system 10 functionalities such a reporting, alert management, etc.

It should be noted that various changes and modifications to the presently preferred embodiments described herein will be apparent to those skilled in the art. Such changes and modifications may be made without departing from the spirit and scope of the present invention and without diminishing its attendant advantages.

1. A system comprising:
a patient data set stored in a patient database, the patient data set including at least a patient identifier, an appointment date, an appointment type, and required appointment prerequisites;
a healthcare provider data set stored in a healthcare provider database, the healthcare provider data set including a plurality of physician types associated with the healthcare providers of the healthcare practice, a plurality of appointment options for each physician type, and revenue of a plurality of healthcare visits;
a financial data set stored in a financial database, the financial data set including a plurality of visit costs, a plurality of payment methods, a plurality of insurance types, and a plurality of payment outcomes;
a controller in communication with each of the patient database, the healthcare provider database, and the financial database; and
a memory coupled to the controller, wherein the memory is configured to store program instructions executable by the controller such that, in response to executing the program instructions, the controller:
calculates a visit value score associated with a scheduled future healthcare visit for a patient end user, wherein the visit value score is based on a visit input data set comprising at least one data element from each of the patient data set, the healthcare provider data set, and the financial dataset;
wherein, when the calculated visit value score is below a pre-defined score limit, the controller:
identifies one or more of the data elements in the visit input date set that may be altered by an action to be taken by the patient end user or a healthcare provider end user to improve the calculated visit value score; and
notifies either the patient end user or the healthcare provider end user of the action to be taken with instructions to take the action.

2. The system of claim 1 wherein, in response to executing the program instructions, the controller further:
receives notice that at least one data element in the visit input data set has been revised;
calculates a revised visit value score associated with the scheduled future healthcare visit for the patient end user based on a revised visit input data set; and
wherein, when the calculated revised visit value score is below the pre-defined score limit, the controller:
identifies one or more of the data elements in the revised visit input date set that may be altered by an action to be taken by the patient end user or a healthcare provider end user to improve the calculated visit value score; and
notifies either the patient end user or the healthcare provider end user of the action to be taken with instructions to take the action.

3. The system of claim 1, wherein the visit value calculated is displayed upon a graphical user interface.

4. The system of claim 1, wherein the visit value calculated is utilized at least in part to generate a report.

5. The system of claim 1, wherein the notification generated by the system is an email, test message, or SMS message.

6. The system of claim 1, wherein at least one data element from the patient data set, the healthcare provider data set, or the financial dataset is supplied from an external data source.

7. The system of claim 2, wherein the action to be taken with instructions to take the action are displayed upon a graphical user interface.

8. The system of claim 2, wherein the notification of action to be taken with instructions to take is an email, test message, or SMS message.

9. A method of generating a healthcare visit value, comprising:
storing a patient data set in a patient database, the patient data set including at least a patient identifier, an appointment date, an appointment type, and required appointment prerequisites;
storing a healthcare provider data set in a healthcare provider database, the healthcare provider data set including a plurality of physician types associated with the healthcare providers of the healthcare practice, a plurality of appointment options for each physician type, and revenue of a plurality of healthcare visits;
storing a financial data set stored a financial database, the financial data set including a plurality of visit costs, a plurality of payment methods, a plurality of insurance types, and a plurality of payment outcomes;
a controller communicating with each of the patient database, the healthcare provider database, and the financial database; and
a memory coupled to the controller, wherein the memory stores program instructions executable by the controller such that, in response to executing the program instructions, the controller:
calculates a visit value score associated with a scheduled future healthcare visit for a patient end user, wherein the visit value score is based on a visit input data set comprising at least one data element from each of the patient data set, the healthcare provider data set, and the financial dataset;
wherein, when the calculated visit value score is below a pre-defined score limit, the controller:
identifies one or more of the data elements in the visit input date set that may be altered by an action to be taken by the patient end user or a healthcare provider end user to improve the calculated visit value score; and
notifies either the patient end user or the healthcare provider end user of the action to be taken with instructions to take the action.

10. The method of claim 9 wherein, in response to executing the program instructions, the controller further:
receives notice that at least one data element in the visit input data set has been revised;
calculates a revised visit value score associated with the scheduled future healthcare visit for the patient end user based on a revised visit input data set; and
wherein, when the calculated revised visit value score is below the pre-defined score limit, the controller:
identifies one or more of the data elements in the revised visit input date set that may be altered by an action to be taken by the patient end user or a healthcare provider end user to improve the calculated visit value score; and
notifies either the patient end user or the healthcare provider end user of the action to be taken with instructions to take the action.

11. The method of claim 9, wherein the visit value calculated is displayed upon a graphical user interface.

12. The method of claim 9, wherein the visit value calculated is utilized at least in part to generate a report.

13. The method of claim 9, wherein the notification generated by the system is an email, test message, or SMS message.

14. The method of claim 9, wherein at least one data element from the patient data set, the healthcare provider data set, or the financial dataset is supplied from an external data source.

15. The method of claim 10, wherein the action to be taken with instructions to take the action are displayed upon a graphical user interface.

16. The method of claim 10, wherein the notification of action to be taken with instructions to take is an email, test message, or SMS message.
